(19) United States
(12) Patent Application Publication
(10) Pub. No.: US 2024/0182848 A1
(43) Pub. Date: Jun. 6, 2024

(54) SULFAMETHOXAZOLE-DEGRADING PSEUDOMONAS SILESIENSIS STRAIN, AND APPLICATION THEREOF

(71) Applicant: CHINESE RESEARCH ACADEMY OF ENVIRONMENTAL SCIENCES, Beijing (CN)

(72) Inventors: Xiaohui LIU, Beijing (CN); Shaoyong LU, Beijing (CN); Jing CHEN, Wuhan (CN); Jiamin XU, Beijing (CN); Jing WANG, Wuhan (CN); Dongru QIU, Wuhan (CN); Yongqiang WANG, Beijing (CN); Xiaochun GUO, Beijing (CN); Ying LIU, Beijing (CN)

(21) Appl. No.: 18/551,841

(22) PCT Filed: Jun. 24, 2022
(86) PCT No.: PCT/CN2022/101052

§ 371 Date: Sep. 21, 2023

(30) Foreign Application Priority Data
Jul. 13, 2021 (CN) 202110787119.8

(51) Int. Cl. C12N 1/20; B09C 1/10; C02F 3/34; C02F 11/02; C12R 1/38

(52) U.S. Cl. CPC C12N 1/205; B09C 1/10; C02F 3/34; C02F 11/02; C02F 2101/40; C12R 2001/38

(57) ABSTRACT

The present disclosure relates to the field of microorganisms, and specifically relates to a sulfamethoxazole-degrading Pseudomonas silesiensis strain and an application thereof. The strain is preserved at the China Center for Type Culture Collection, has the preservation number: CCTCC No: M2021338, and was preserved on 6 Apr. 2021. The present strain has a strong ability to degrade sulfamethoxazole, and SMX removal of approximately 75% or more may be achieved in an R2A culture medium; additionally, said strain is also a type of electrochemically active bacterium.

### CROSS-REFERENCE TO RELATED APPLICATIONS

This application is a national stage for International Application No. PCT/CN2022/101052, filed on Jun. 24, 2022, which claims the priority benefit of Chinese patent application No. 202110787119.8, filed on Jul. 13, 2021, and titled “SULFAMETHOXAZOLE-DEGRADING PSEUDOMONAS SILESIENSIS STRAIN AND APPLICATION THEREOF”, the content of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on 30 Aug. 2023, is named SP23704116US_Sequence Listing.txt and is 1,572 bytes in size.

### TECHNICAL FIELD

The present disclosure relates to the field of microorganisms, and specifically relates to a sulfamethoxazole-degrading Pseudomonas silesiensis strain and an application thereof.

### BACKGROUND

Sulfamethoxazole (SMX), as a typical class of sulfonamide antibiotics, can interrupt synthesis of bacteria, thereby inhibiting bacterial growth. Bioelectrochemical technology mainly improves the effect of SMX removal by strengthening the regulation of microorganisms in constructed wetlands. However, the bioelectrochemically enhanced constructed wetland is a relatively complex system, and it is difficult to thoroughly understand the metabolic process of SMX in the system, which is not beneficial to the further understanding of the SMX degradation process in the constructed wetlands. At present, the research on SMX-degrading bacteria in constructed wetlands is very limited, and the research that has been carried out is mainly focused on aspects such as the screening of, the analysis of intermediate products from, degradation kinetics and co-metabolism of the SMX-degrading bacteria in active sludge or sediment contaminated by antibiotics, or the like. However, there are few studies on the protein and molecular mechanism for microbial degradation. In view of this, the present disclosure is proposed.

### SUMMARY

The present disclosure relates to an isolated Pseudomonas silesiensis strain, which is deposited in the China Center for Type Culture Collection on Apr. 6, 2021, with a deposit number of CCTCC No: M2021338.

The strain, named F6a, was isolated from constructed wetlands and forms a large colony which is smooth, translucent, slightly raised, neat edge, wavy, medium size and in a milky-white color, on a R2A solid medium. Under a microscope, the bacterium was long rod-shaped, and some of the bacteria showed a filamentous shape. The bacterial morphology was observed by a transmission electron microscope, and the F6a strain was mainly in a form of long rod or oval, with flagella. Extracellular polymers and substances similar to extracellular vesicles were found around the F6a strain, and both extracellular polymers and vesicles contain a variety of biologically active substances, which is useful for strengthening the cooperation between cells, thereby improving the degradation efficiency of pollutants.

According to another aspect of the present disclosure, the present disclosure also relates to a composition comprising the strain as described above.

According to another aspect of the present disclosure, the present disclosure also relates to a method for culturing the strain as described above, comprising culturing the Pseudomonas silesiensis strain in a R2A medium.

According to still another aspect of the present disclosure, the present disclosure also relates to use of the strain as described above or the composition as described above in degrading sulfamethoxazole.

### BRIEF DESCRIPTION OF THE DRAWINGS

In order to more clearly illustrate the specific embodiments of the present disclosure or the technical solutions, the accompanying drawings that need to be used in the description of the specific embodiments are briefly described in the below. Obviously, the accompanying drawings in the following description are some embodiments of the present disclosure. Those persons skilled in the art can obtain other drawings based on these drawings without any creative work.

FIG. 1 shows the removal rate of SMX of 47 strains in MSM medium and R2A medium under initial SMX of 10 mg/L according to an embodiment of the present disclosure.

FIG. 2 shows (a) a colony diagram and (b) a microscopic diagram of the morphological characteristics of the F6a strain in R2A solid medium containing SMX of 10 mg/L according to an embodiment of the present disclosure.

FIG. 3 shows the transmission electron micrographs of the F6a strain according to an embodiment of the present disclosure.

FIG. 4 shows the phylogenetic tree of the F6a strain according to an embodiment of the present disclosure.

FIG. 5 shows the degradation effect of the F6a strain under different initial concentrations of SMX according to an embodiment of the present disclosure.

FIG. 6 shows the removal rate of TOC during the SMX degradation under initial SMX of 10 mg/L according to an embodiment of the present disclosure.

FIG. 7A-7G show the three-dimensional fluorescence spectrogram during the SMX degradation by the F6a strain according to an embodiment of the present disclosure.

FIG. 8 shows (a) classification of antibiotic resistance genes of the F6a strain and (b) annotation of drug-resistant genes of whole genome of the F6a strain by the CARD database, in which different colors in the ring diagram represent classification of different antibiotic resistance ontology (ARO) and ring areas represent numbers of classified genes of the measured genome in the classification and their relative proportions according to an embodiment of the present disclosure.

FIG. 9 shows the PCR electrophoresis diagram of sul1, sul2, sul3, sulA, int1 and int2 genes according to an embodiment of the present disclosure.

FIG. 10 shows (a) protein information and (b) distribution of peptide fragment number according to an embodiment of the present disclosure.

FIG. 11 shows the GO functional annotation of differential proteins according to an embodiment of the present disclosure.

FIG. 12 shows the KEGG pathway annotation of differential proteins according to an embodiment of the present disclosure.

FIG. 13 shows the COG functional annotation of differential proteins according to an embodiment of the present disclosure.

FIG. 14 shows the PFAM functional annotation of differential proteins according to an embodiment of the present disclosure.

FIG. 15 shows the PCR electrophoresis diagram of sadA, sadB and sadC genes according to an embodiment of the present disclosure.

FIG. 16 shows the metabolic pathways of SMX degrading by the F6a strain according to an embodiment of the present disclosure.

FIG. 17 shows the mass spectrum of intermediate products in the SMX degradation process according to an embodiment of the present disclosure.

The Pseudomonas silesiensis strain provided in the present disclosure is named F6a (i.e., Pseudomonas silesiensis strain F6a) and is deposited in the China Center for Type Culture Collection located on Wuhan University, Wuhan, China on Apr. 6, 2021, with a deposit number of CCTCC No: M2021338. The strain F6a was detected to be viable by the depository authority on Apr. 13, 2021.

### DETAILED DESCRIPTION OF THE EMBODIMENTS

Reference will now be made in detail to embodiments of the present disclosure, and one or more examples of which are described below. Each example is provided by way of explanation, not limitation of the present disclosure. In fact, it will be obvious to those persons skilled in the art that various modifications and variations may be made in the present disclosure without departing from the scope or spirit of the present disclosure. For example, features illustrated or described as a part of one embodiment may be used on another embodiment to generate a still further embodiment.

Unless otherwise defined, all technical terms and scientific terms used herein have the same meaning as commonly understood by one of ordinary skill in the technical field of the present disclosure. The terms used herein in the description of the present disclosure are for the purpose of describing specific embodiments only, and are not intended to limit the present disclosure. As used herein, the term “and/or” includes any and all combinations of one or more of the related listed items.

The present disclosure relates to an isolated Pseudomonas silesiensis strain, which is deposited in the China Center for Type Culture Collection on Apr. 6, 2021, with a deposit number of CCTCC No: M2021338.

The strain of the present disclosure has a strong ability to degrade sulfamethoxazole and can remove about 75% or more of SMX in the R2A medium; meanwhile, the strain is also an electrochemically active bacterium.

The present disclosure seeks protection for the Pseudomonas silesiensis strain with the deposit number as described above and mutant strains having mutations in a moderate range and retaining a strong SMX degradation ability, for example, at least 50%, or 60%, or 70%, or 80%, or 90%, or 95%, or 95% of the SMX degradation ability of the F6a strain.

The called “mutant strain of the Pseudomonas silesiensis strain” refers to a Pseudomonas silesiensis strain whose genome is highly similar to the genome of the F6a strain. In the present disclosure, the expression of “Pseudomonas silesiensis strain of the present disclosure” covers said mutant strains. The mutant strains may be defined by having a 16S rDNA homology of equal to or more than 99% (such as, 99.1%, 99.2%, 99.3%, 99.4%, 99.5%, 99.6%, 99.7%, 99.8% or 99.9% of homology) with the 16S rDNA of the F6a strain shown in SEQ ID NO: 1, or can also be defined by a highly similar genome. The mutant strains can also be strains in which the genome is mutated. Compared to the genome of the F6a strain, the genome of a mutant strain of the Pseudomonas silesiensis strain contains at most 150 mutation events, for example, at most 140, 130, 120, 110, 100, 90, 80, 70, 60, 50, 40, 30 or 20 mutation events. The mutation events are defined as single nucleotide polymorphisms (SNPs) or INDELs (i.e., insertions, deletions, and the combination of both). The number of mutation events is determined as follows: taking the genome of the F6a strain as a control, identifying mutation events presented in the genome of the mutant strain, in which each type of mutation event (i.e., SNP or INDEL) represents one mutation event, that is, for example, the insertion of a sequence containing several nucleotides is considered as one mutation event. In this context, the genomic sequence of the mutant strain of the present disclosure is defined by the number of mutation events contained with respect to the genomic sequence of the F6a strain, or otherwise additionally it is defined by an identity percent with respect to the genomic sequence of the F6a strain. The identity percent herein means the percent of sequences found in the genome of one strain that are present in the genome of another strain. Specifically, the identity percent herein includes a) the percent of sequences found in the genome of the F6a strain that are present in the genome of the mutant strain, or b) the percent of sequences found in the genome of the mutant strain that are present in the genome of the F6a strain. Therefore, for a mutant strain that differs from the F6a strain merely by including one or more insertions or one or more deletions, the identity percent between the genome of the mutant strain and the genome of the F6a strain is 100%, because entire genome sequences of one strain is totally found in the genome of the other strain. In a particular embodiment, the genomic sequence of the mutant strain of the present disclosure, defined by the number of mutation events, has an identity percent of at least 90%, at least 91%, at least 92%, at least 93%%, at least 94%, at least 95%, at least 96%, at least 97%, at least 98%, at least 99%, at least 99.1%, at least 99.2%, at least 99.3%, at least 99.4%%, at least 99.5%, at least 99.6%, at least 99.7%, at least 99.8%, at least 99.9%, at least 99.92%, at least 99.94%, at least 99.96%, at least 99.98%, or at least 99.99%% in relative to the genomic sequence of the F6a strain. The identity percent means the percent of sequences found in the genome of one strain that are present in the genome of another strain. The identity is determined by comparing their full lengths of the two genome sequences (i.e., global comparison) and may be calculated by using any program based on the Needleman-Wunsch algorithm.

In the process of practical application, in view of reasons, such as transportation that may be required, or the like, it is necessary to amplify and culture the Pseudomonas silesiensis strain into a form of a composition, especially a microbial preparation, to expand its application range.

Composition of the present disclosure may be a pure culture or a mixed culture. Therefore, the present disclosure defines a pure culture as a culture in which all or substantially all of the culture consists of the same Pseudomonas silesiensis strain of the present disclosure. In an alternative form, a mixed culture is defined as a culture including several kinds of microorganisms, in particular including several kinds of bacterial strains, in which the Pseudomonas silesiensis strain of the present disclosure is included.

The composition may be prepared into a form of liquids, or frozen or dry powders; or expressed in a form of preparations commonly used in this industry, such as granules, suspensions, wettable powders, emulsions or liquids.

In some embodiments, the composition includes an adjuvant.

In some embodiments, the adjuvant includes one or more of sodium dodecylbenzenesulfonate, sodium butylnaphthalenesulfonate, trehalose, glycerin, sodium lignosulfonate, polycondensate of sodium alkylnaphthalenesulfonate, nicotinic acid, alcohol, buffer salt, sodium chloride, amino acid, vitamins, protein, polypeptide, polysaccharide or monosaccharide, yeast extract, white carbon black, tea saponin, and skimmed milk.

In some embodiments, when the composition is in the form of frozen or dried powders, the composition further includes a solid carrier;
the solid carrier includes one or more of peat, turf, talc, lignite, pyrophyllite, montmorillonite, alginate, filter press slurry, sawdust, perlite, mica, silica, quartz powder, calcium-based bentonite, vermiculite, kaolin, light calcium carbonate, diatomaceous earth, medical stone, calcite, zeolite, white carbon black, fine sand or clay.

The total content of the adjuvants may be 0 wt % to 80 wt %, such as 0.5 wt %, 1 wt %, 5 wt %, 10 wt %, 20 wt % or 50 wt %. The content of the carrier is a value by subtracting the contents of active ingredients and adjuvants from 100 wt %.

The present disclosure also relates to a method for culturing the strain as described above, including culturing the Pseudomonas silesiensis strain in a R2A medium.

The present disclosure also relates to use of the strain as described above or the composition as described above in degrading sulfamethoxazole.

In some embodiments, the sulfamethoxazole is present in water, soil or sludge contaminated by antibiotics.

The water may be from rivers, lakes, seas, groundwater, or the like. Further, the water may also be municipal wastewater, agricultural wastewater, or industrial wastewater such as food production wastewater, dye wastewater or the like.

The sludge generally refers to mud located in river courses or other water sources.

In some embodiments, the strain or the composition cooperates with a microbial fuel cell to degrade sulfamethoxazole.

The present disclosure also relates to a bioelectrochemically coupled constructed wetland system, which contains the strain as described above or the composition as described above.

In some embodiments, the system further contains a microbial fuel cell, and the strain or the composition cooperates with the microbial fuel cell to degrade sulfamethoxazole.

Embodiments of the present disclosure will be described in detail below in combination with examples.

### EXAMPLES

### Example 1. Test Materials and Methods

### 1.1 Screening and Isolation of SMX-Degrading Bacteria

### 1.1.1 Mediums for Experiments.

According to the experimental requirements, the R2A medium and minimal mineral salt medium (MSM) for strains are shown in Table 1. The mediums were adjusted to a pH of 7.0 to 7.2. 15 g/L of agar was added to the solid medium. All the mediums were autoclaved at 121ºC for 20 minutes, followed by cooling to 55° C. to 60° C. and adding 50% sterile glucose solution for later use.

### 1.1.2 Enrichment, Isolation and Purification of SMX-Degrading Bacteria.

The matrix samples in cathode and anode of a direct current-enhanced constructed wetland (EC-CW) device and a microbial fuel cell-enhanced constructed wetland (MFC-CW) device were collected respectively. Each sample was rinsed with sterile water. The microorganisms in the samples were washed down by vortex shaking, and shook well. Under the condition of energization at 0.4 V, in an ultra-clean workbench (in which samples in lower system was placed in an anaerobic aseptic operation bench and samples in upper system was placed in a vertical wind ultra-clean workbench), 1 mL of bacterial solutions washed down from various samples were respectively inoculated in 100 mL of the R2A medium containing different concentrations of SMX sterilized by filtration, and cultured in a constant temperature shaker at 28° C. and 200 rpm/min for 24 hours, in which the concentrations of SMX were respectively 1 mg/L, 10 mg/L and 100 mg/L. The enriched culture was diluted with sterile water by multiples of 10⁻¹, 10⁻², 10⁻³, 10⁻⁴ and 10⁻⁵. 0.1 mL of individual diluted cultures were spread on the R2A solid medium containing corresponding concentrations of SMX in plates. The plates were placed upside-down in a constant temperature incubator at 28° C. and cultured for 24 to 36 hours, during which the growth of colonies was observed. Single colonies having different morphologies were remarked, and the single colonies were picked up with sterile toothpicks for streaking and isolation, until the single colonies having basically the same colony characteristics were obtained.

TABLE 1
Mediums for experiments

| Mediums | Component | Content | | Component | Content | |
|---|---|---|---|---|---|---|
| R2A | Tryptone | 0.5 | g · L⁻¹ | Sodium pyruvate | 0.3 | g · L⁻¹ |
| medium | Yeast extract | 0.5 | g · L⁻¹ | K₂HPO₄ | 0.3 | g · L⁻¹ |
| | Starch | 0.5 | g · L⁻¹ | MgSO₄•7H₂O | 0.024 | g · L⁻¹ |
| | Asein acids | 0.5 | g · L⁻¹ | 50% glucose (sterilized | 0.1% | (V/V) |
| | Hydrolysate | | | by filtration) | | |
| MSM | NaH₂PO₄ | 2.8 | g · L⁻¹ | CoCl₂•6(H₂O) | 0.02 | mg · L⁻¹ |
| medium | KH₂PO₄ | 1 | g · L⁻¹ | FeSO₄•7H₂O | 0.2 | mg · L⁻¹ |
| | (NH₄)₂SO₄ | 0.5 | g · L⁻¹ | ZnSO₄•7H₂O | 0.01 | mg · L⁻¹ |
| | MgCl₂ | 53 | mg · L⁻¹ | MgCl₂•4H₂O | 0.003 | mg · L⁻¹ |
| | Ca(NO₃)₂•4H₂O | 50 | mg · L⁻¹ | Na₂MoO₄•2H₂O | 0.003 | mg · L⁻¹ |
| | EDTA-2Na | 0.5 | mg · L⁻¹ | NiCl₂•6H₂O | 0.002 | mg · L⁻¹ |
| | H₃BO₃ | 0.03 | mg · L⁻¹ | CuCl₂•2H₂O | 0.001 | mg · L⁻¹ |

### 1.1.3 Screening of SMX-Degrading Bacteria

The minimal mineral salt medium (MSM) and the R2A medium containing additional carbon and nitrogen sources were respectively used to screen SMX-degrading bacteria. 1 mL of the enriched SMX-resistant bacterial solution was added to 100 mL of the MSM liquid medium containing 1 mg/L SMX, 10 mg/L SMX or 100 mg/L SMX respectively. Under the condition of energization, the bacterial solution was cultured in a constant temperature shaker at 28° C. and 200 rpm/min. The concentration of SMX in the medium was detected at different times. The growth of the microbial flora was observed by detecting the OD₆₀₀ value. After the microbial flora grew significantly, the corresponding solid medium was used to isolate and identify the screened bacteria, thereby further screening out the SMX-degrading bacteria. The specific method is as follows.

100 μL of the bacterial solution was taken for serial dilution. After that, 100 μL of individual diluted solutions were taken and spread on the MSM medium or the R2A medium containing corresponding concentrations of SMX in plates. The plates were placed upside-down in a constant temperature incubator at 28° C. and cultured for 24 to 36 hours, during which the growth of colonies was observed. After the colony grew to an appropriate size, the plates in serial dilution and with a suitable colony growth density were selected. According to different characteristics of the bacteria, including colony size, color, shape, edge, transparency, protrusion, consistency or the like, single clones were picked up with sterile toothpicks and inoculated into the MSM liquid medium and the R2A liquid medium respectively containing the corresponding concentrations of SMX, followed by culturing in a constant temperature shaker at 28° C. and 200 rpm/min for 24 to 36 hours. The OD 600 value was monitored, and the bacteria with obvious growth were identified through the same method as described above. The identified strains were stored in 20% glycerol and stored at −80° C. for future use.

### 1.2 Identification of SMX-Degrading Bacteria

### 1.2.1 Morphological Identification

The morphological characteristics of the strains include colony size, color, shape, edge, transparency, protrusion, consistency, or the like. The bacterial morphology was observed under a microscope through bacterial fixation and staining. The sub-microstructure or ultrastructure of the strains were further observed by using the transmission electron microscope (HT7700, Hitachi). The specific method for microscopic observation includes: 1) making a smear. A clean slide was taken and a circle with a diameter of about 1.5 cm was drawn using a pencil on the back of the slide as a mark of the smear range. On an aseptic operation bench, bacterial solution was dropped into the circle and slightly tilted to spread over and within the circle. The slide was passed forth and back three times over the outer flame of an alcohol lamp to fix the bacterial film and it is necessary to not overheat to sear the bacterial film. 2) Staining. Methylene blue staining solution was dropped on the bacterial film and stained for 1 minute. The remaining dye was washed by gently flowing water through the bacterial film. 3) Microscopic examination. The bacterial morphology was observed under 100× oil lens of an optical microscope and pictures were taken for preservation.

1.2.2 Identification of 16s rRNA Gene of Bacteria

Full-length sequence of 16S rDNA of bacterial strain was amplified by the polymerase chain reaction with primers 27F (5′-AGAGTTTGATCCTGGCTCAG-3′) and 1492R (5′-TACGGCTACCTTGTTACGACTT-3′) based on the colony PCR reaction. The PCR products were detected by 1% agarose electrophoresis. After the detection with correct identification, the PCR products were purified by using the PCR product purification kit (omega, USA) and then subjected to bidirectional sequencing. The spliced full-length sequence was uploaded to BLASTN on NCBI (http://www.ncbi.nlm.nih.gov/BLAST) for comparison. A strain with the highest similarity was selected as a reference strain, and the category of said bacterial strain was determined. The PCR reaction system and the reaction procedure are respectively shown in Table 2 and Table 3.

TABLE 2
The amounts of reagents in PCR reaction system

| Name | Amount (μL) |
|---|---|
| 2 × Es Taq master Mix | 25 |
| Bacteria solution | 1.0 |
| Primer1 27F: | 1.0 |
| Primer2 1492R: | 1.0 |
| dd H₂O | Up to 50 |

TABLE 3
PCR reaction procedure

| | Reaction | Reaction |
|---|---|---|
| Reaction Procedure | Temperature (° C.) | Time (min) |
| \textcircled{1}Pre-denaturation | 94 | 5 |
| \textcircled{2}Denaturation | 94 | 0.5 |
| \textcircled{3}Annealing | 55 | 0.5 |
| \textcircled{4}Extension | 72 | 1 |

Repeat steps \textcircled{1} to \textcircled{3} for 30 cycles

| | | |
|---|---|---|
| \textcircled{5}Extension | 72 | 10 |

### 1.3 Study on Growth and Degradation Characteristics of Strain

A single colony of the F6a strain in the solid medium was picked up, inoculated into the liquid medium under the aseptic conditions and cultured at 28° C. under shaking at 200 rpm/min for 48 hours. After said bacterial strain grew to the logarithmic phase, the bacterial solution was placed in a sterilized centrifuge tube, centrifuged at 10000 rpm/min for 5 minutes, followed by discarding the supernatant and collecting the bacteria precipitate. The bacteria precipitate was washed with sterile PBS buffer and made into bacterial inoculum.

The bacterial inoculum at 1% inoculum amount was added to the R2A medium containing different concentrations of SMX (i.e., 1 mg/L, 5 mg/L, 10 mg/L, 50 mg/L, 80 mg/L and 100 mg/L). The bacterial solution was placed in a constant temperature shaker at 28° C. and cultured in the dark with shaking at 200 rpm/min. Each sample was repeated in triplicate. Samples were taken at 24 h, 48 h, 96 h, 120 h, 144 h, 264 h and 336 h respectively for determining the SMX concentration and the OD₆₀₀. The effects of the initial concentration of SMX on the growth of the strain and the degradation of SMX were investigated.

The degradation kinetics of SMX was fitted with a pseudo-first-order kinetic equation according to the following formula.

InC=InC₀+kt

in which, k represents the rate constant of pseudo-first-order kinetics, in h⁻¹; C represents the concentration of SMX at the time of degradation t (h), in mg/L; C₀ represents the initial concentration of SMX, in mg/L; and the half-life is ln 2/k.

### 1.4 Intermediate Products of SMX and Analysis of Mineralization Degree

The F6a strain at 1% inoculum amount was inoculated to the R2A liquid medium containing 10 mg/L of SMX, and cultured in the dark at 28° C. with shaking at 200 rpm/min. The intermediate products of SMX were measured by UPLC-QTOF-MS at 24 h, 48 h, 96 h, 120 h, 144 h, 264 h and 336 h respectively when the F6a strain degraded SMX. Meanwhile, total organic carbon (TOC) was measured by the TOC-L CPH (Shimadzu, Japan) and three-dimensional fluorescence was measured by the Hitachi F-7000, so as to detect the mineralization and metabolic pathways of SMX during the degradation process.

The three-dimensional fluorescence was measured by diluting the solution in 10 times to reduce the internal filter effect. The light source of the fluorescence spectrophotometer is a 150 W xenon lamp, the voltage of the photomultiplier tube is 700 V, the excitation wavelength is 200 nm to 450 nm, the emission wavelength is 250 nm to 600 nm, the slit width of both excitation wavelength and emission wavelength is 5 nm, and the scanning speed is 12000 nm/min. The fluorescence spectrophotometer was automatically calibrated according to the Raman signal and normalized with quinine sulfate in unit. Raman scattering and Rayleigh scattering were eliminated by subtracting the blank water sample, manually resetting or the like.

### 1.5 Whole Genome Sequencing

### 1.5.1 DNA Extraction.

DNA was extracted according to the method of Wizard® Genomic DNA Purification Kit (Promega), and was quantified through the TBS-380 fluorescence instrument (Turner BioSystems Inc. Sunnyvale, CA).

### 1.5.2 Illumina Library Construction and Sequencing.

Library was prepared in strict accordance with the method of the NEXTflexTMRapid DNA-Seq kit, and paired-end sequencing (2×150 bp) was performed on the Illumina HiSeq X Ten instrument.

### 1.5.3 Genome Assembly, Gene Prediction and Annotation.

Bioinformatics analysis was performed on the Illumina platform. The sequencing image signal was converted into a text signal through CASAVA base calling and stored in fastq format. In order to ensure the accuracy of assembly, raw reads were cut for quality according to the research method of Zhu Manli. Clean data was spliced through the SOAP denovo 2 Assembly software to obtain optimal assembly results. The coding sequences (CDS), tRNA, and rRNA of the genome were respectively predicted through Glimmer, tRNAscan-SE, and Barrnap. Related feature information of genes was annotated by using tools such as BLAST, Diamond, HMMER or the like.

### 1.6 Test Method for Proteomics

### 1.6.1 Protein Extraction.

The operation process was conducted as follows. 1) All the samples in the frozen state were taken out and transferred to the MP oscillating tube. 2) An appropriate amount of extraction buffer including 1% SDS, 200 mM dithiothreitol, 50 mM Tris-HCl in pH 8.8 and protease inhibitors was added and mixed well by vortexing. 3) The mixture was oscillated using a high-throughput tissue grinder for 3 times, with 40 seconds each time. 4) The mixture was incubated at 100° ° C. for 10 minutes and then cooled on ice. 5) The mixture was subjected to lysis on ice for 30 minutes with vortex mixing for 5 to 10 seconds at intervals of 5 minutes. 6) The solution was centrifuged at 4° C. and 12000 g for 20 minutes, and the supernatant was collected. 7) Pre-cooled acetone was added at a ratio of 1:4, and kept at −20° ° C. overnight for precipitation. 8) In the next day, the solution was centrifuged at 4° ° C. and 12000 g for 20 minutes and the supernatant was discarded. 90% pre-cooled acetone was added to the precipitate, mixed well, and centrifuged with the supernatant discarded, which was repeated for 2 times. 9) The precipitate was dissolved with protein lysate including 8M urea, 1% SDS and protease inhibitors. 10) The solution was centrifuged at 4° C. and 12000 g for 20 minutes and the supernatant containing proteins was collected. The total protein of the sample was obtained after the above steps 1) to 10). 11) The total protein was quantified through the Thermo Scientific Pierce BCA kit. The BCA kit was used to formulate the BCA working solution and standard protein solutions of different mass concentrations, which are 0 mg/mL, 0.125 mg/mL, 0.250 mg/mL, 0.500 mg/mL, 0.750 mg/mL, 1.000 mg/mL, 1.500 mg/mL and 2.000 mg/mL, respectively. 2 μL of each sample was mixed with 18 μL of water and 200 μL of BCA working solution was added. The resulting solution was vortex mixing well, reacted at 37ºC for 30 minutes, and the absorbance was read at 562 nm. 12) 15 μg of each sample was loaded for the SDS-PAGE electrophoresis.

### 1.6.2 Enzymatic Alkylation and Labeling.

To 100 μg of protein sample, a lysis solution was supplemented to the volume of 90 μL. Tris(2-carboxyethyl) phosphine as a reducing agent was added at a final concentration of 10 mmol/L and reacted at 37° C. for 60 minutes. Iodoacetamide was added at a final concentration of 40 mmol/L and reacted in the dark at room temperature for 40 minutes. Pre-cooled acetone was added to each tube at a volume ratio of 6:1 of acetone to the sample, stilled at −20° ° C. for 4 hours for precipitation and centrifuged at 10000 g for 20 minutes, and the precipitate was collected. The protein precipitate was fully dissolved with 50 mmol/L of triethylammonium bicarbonate (TEAB). Trypsin was added at a mass ratio of 1:50 of the enzyme to the protein sample for enzymatic hydrolysis at 37° C. overnight. Tandem mass tag reagent (TMT, Thermofisher) at −20° C. was warmed to the room temperature, followed by adding acetonitrile, vortex and centrifugation. A tube of TMT reagent was added for every 100 μg of the protein sample, and then incubated at room temperature for 2 hours. Hydroxylamine was added for reaction at room temperature for 15 minutes. After that, equal amounts of labeled products were mixed in one tube, and dried in a vacuum concentrator.

### 1.6.3 Protein Identification.

One-dimensional separation by reversed-phase liquid chromatography and liquid chromatography-tandem mass spectrometry was conducted. The polypeptide sample redissolved in buffer was loaded to the Ultra Performance Liquid Chromatography (UPLC) and subjected to the high pH liquid phase separation in the reverse phase C18 column. Phase A was 2% acetonitrile, which was adjusted to pH of 10 with ammonia water. Phase B was 80% acetonitrile, which was adjusted to pH of 10 with ammonia water. The UV detection wavelength was 214 nm. The volume flow rate was 200 μL/min. The elution time was 66 minutes. A total of 20 fractions were collected according to the peak shape and time, which were combined into 10 fractions, and concentrated by centrifugation in vacuo. The second-dimension separation was analyzed by the nanoliter liquid chromatography-tandem mass spectrometry (i.e., Easy-nLC 1200 in combination with Q Exactive mass spectrometer). Peptide fragments were dissolved in buffer for loading to the mass spectrometry. After loading, the peptide fragments were separated by a C18 chromatographic column in 75 μm×25 cm (Thermo, USA) for 120 minutes, with a volume flow rate of 300 μL/min. The gradient elution for EASY-nLC liquid chromatography was carried out with 2% acetonitrile containing 0.1% formic acid, as phase A and 80% acetonitrile containing 0.1% formic acid as phase B. Automatic switching was performed between the primary mass spectrometry (MS) acquisition and the secondary mass spectrometry (MS/MS) acquisition, with 70 K and 35 K of mass spectrometry resolution respectively. A full scan was performed in the primary mass spectrometry (MS), with mass to charge ratio (i.e., m/z) of 350 to 1300. The top 20 parent ions were selected for secondary fragmentation, and the dynamic exclusion time was 18 seconds.

### 1.6.4 Library Searching for Proteins.

The original raw file obtained by the mass spectrometer was analyzed by the ProteomeDiscoverer™ Software 4.5 (AB Sciex) server. Proteins were identified by using the human SwissProt_2014_08. fasta sequence database. The proteins of corresponding species were searched by using the Uniprot database (www.uniprot.org).

NR or Swiss-Prot functional annotation for proteins was performed by using the DIAMOND (v0.8.37.99) software. Pfam functional annotation for proteins was performed by using the HMMER (3.1b2) software. Subcellular location annotation for proteins was performed by using the MultiLoc2 software. GO functional annotation for proteome was performed by using the BLAST2GO (2.5.0) software. KEGG functional annotation for proteome was performed by using the KOBAS (2.1.1) software. Differential expression level was analyzed by using the R Project. GO enrichment for protein set analysis was performed by using the goatool (s 0.6.5). KEGG enrichment for protein set analysis was performed by using the Python.

The false discovery rate (FDR) of peptide fragment identification during the library searching was set as FDR≤0.01. Protein contains at least one unique peptide. A total of 3440 proteins were detected. P-value of significant difference between samples and the fold change (FC) between groups were calculated by using the t. test function in R language. The criteria for screening proteins with significantly different expression is as follows. p<0.05 and FC>2 represent up-regulated proteins, and p<0.05 and FC<0.5 represent down-regulated proteins.

### 1.6.5 Bioinformatics Analysis.

The gene ontology (GO) database, Cluster of Orthologous Groups of proteins (COG) database, Protein families (PFAM) database were selected to perform the functional cluster analysis for all differential proteins. The Kyoto encyclopedia of gene and genomes (KEGG) database was used to analyze the metabolic pathways involved in differential proteins.

### Example 2. Screening and Identification of SMX-Degrading Bacteria

### 2.1 Screening of SMX-Degrading Bacteria

Each sample was cultured in R2A medium containing SMX at a concentration (i.e., 1 mg/L, 10 mg/L and 100 mg/L, respectively). According to different characteristics of bacteria, including colony size, color, shape, edge, transparency, protrusion, consistency or the like, 132 colonies were selected and a total of 47 strains were identified. Among them, Bacillus thuringiensis strain IAM 12077, Pseudomonas umsongensis strain Ps 3-10 and Bacillus cereus strain IAM 12605 were detected with the most frequency. The 47 strains belong to 28 genera including Bacillus, Pseudomonas, Methylotenera or the like, and belong to 4 phyla including Proteobacteria, Firmicutes, Bacteroidetes, Actinobacteria or the like. Most strains of them belong to the Proteobacteria phylum, and are electrochemically active bacteria, which mainly participate in the degradation of SMX. The results are consistent with the results of high-throughput sequencing. However, the fact that a strain can grow in the medium containing SMX cannot determine whether the strain uses SMX as a carbon source to degrade SMX or the strain is a bacterium resistant to SMX. Therefore, experiments on SMX degradation need to be further developed.

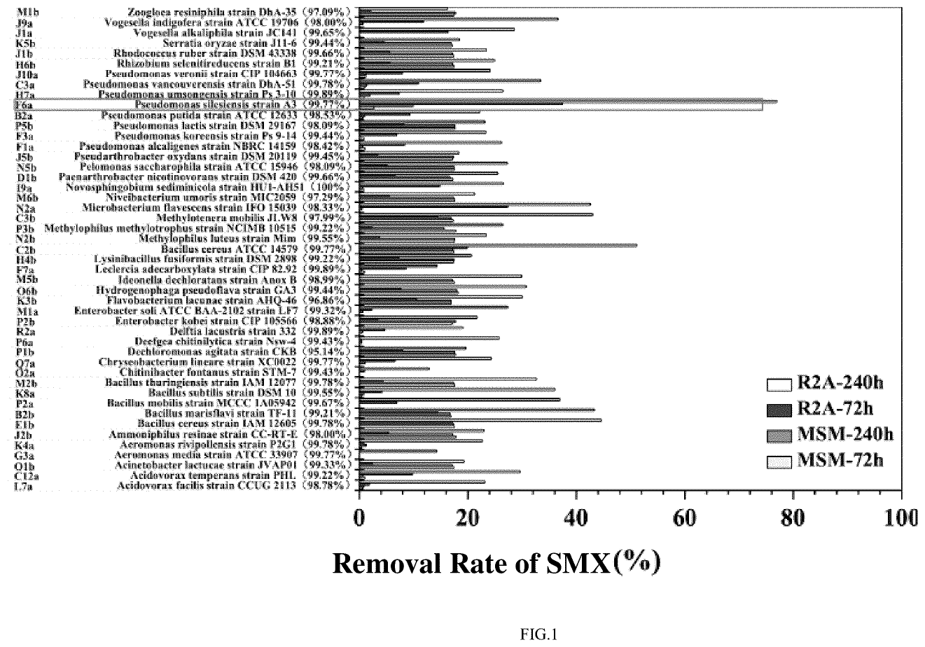

The screened 47 strains were respectively inoculated into the MSM medium containing 10 mg/L of SMX and the R2A medium containing 10 mg/L of SMX, and cultured at 30° C. and 180 rpm for 10 days. The samples were respectively collected at 72 h and 240 h to measure the degradation rate of SMX. The results are shown in FIG. 1. The strains cultured in the MSM medium exhibited a low removal rate of SMX, which is basically less than 20%. However, the strains cultured in the R2A medium exhibited significantly higher removal rate of SMX at 240 h compared to those in the MSM medium. Among them, the F6a strain exhibited the highest removal rate of SMX up to 76.95%, followed by the C2b strain (with 51.16% of removal rate of SMX) and the C3b strain (with 43.01% of removal rate of SMX). According to the removal rate of SMX by the strains in different mediums, co-metabolism is the main way to metabolize SMX by the strains in the system.

The isolated strains belonging to the Pseudomonas genus in the present disclosure do not exhibit ideal removal rate of SMX. However, the Pseudomonas silesiensis strain A3 can achieve 76.95% of removal rate of SMX in the R2A medium. In summary, it may be seen that strains belonging to the same genus or even the same species have quite differences in the utilization degree of sulfonamide antibiotics. Therefore, the F6a strain was selected as the experimental degrading bacterium in the present disclosure to further study the mechanism for the metabolism and molecular regulation of SMX.

### 2.2 Identification of SMX-Degrading Bacteria

### 2.2.1 Morphological Identification

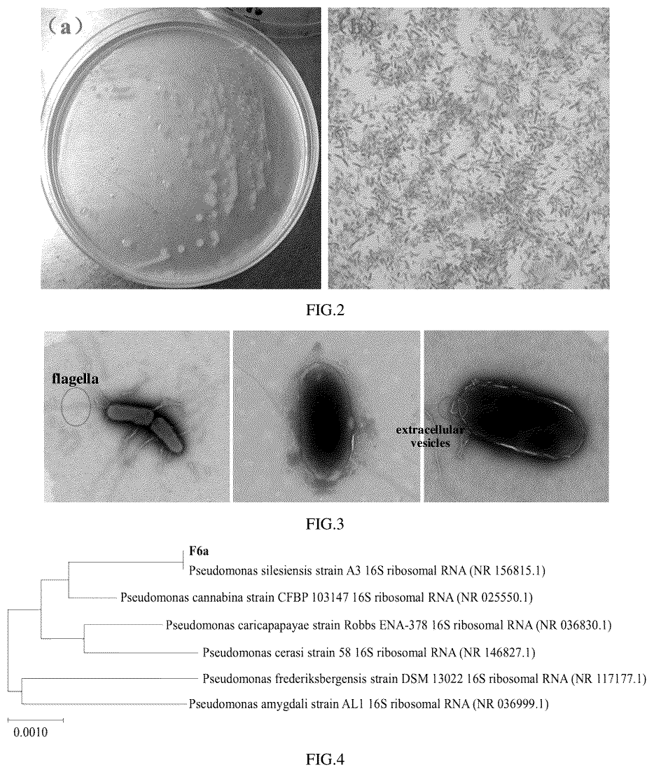

On the R2A solid medium, the F6a strain (i.e., Pseudomonas silesiensis strain A3) formed a large colony which was smooth, translucent, slightly raised, neat edge, wavy, medium size and in a milky-white color (FIG. 2a). Under a microscope, the bacterium was long rod-shaped, and some of the bacteria showed a filamentous shape (FIG. 2b). The bacterial morphology was observed by a transmission electron microscope (FIG. 3), and the F6a strain was mainly in a form of long rod or oval, with flagella. Extracellular polymers and substances similar to extracellular vesicles were found around the F6a strain, and both extracellular polymers and vesicles contain a variety of biologically active substances, which is useful for strengthening the cooperation between cells, thereby improving the efficiency for degrading pollutants.

### 2.2.2 Phylogenetic Tree

The sequencing result of the 16S rDNA sequence was uploaded to NCBI. Through the analysis of BLAST comparison, it was found that the homology between the F6a strain and the Pseudomonas silesiensis strain A3 was 99.77%. Thus, the F6a strain belongs to the Proteobacteria phylum, the Gammaproteobacteria class, the Pseudomonadales order, the Pseudomonadaceae family, the Pseudomonas genus. Meanwhile, the F6a strain is also an electrochemically active bacterium. The 16S rDNA sequences of closely related strains were used to construct a phylogenetic tree based on the Neighbor-Joining method in the MEGA6. As shown in FIG. 4, the F6a strain belongs to the Pseudomonas genus in the molecular phylogenetic taxonomy. Studies have shown that Pseudomonas strains can promote the degradation of SMX in microbial fuel cells (MFC).

### Example 3. Study on SMX Degradation Characteristics of F6a Strain

### 3.1 Study on SMX Degradation Kinetics of F6a Strain

FIG. 5 shows the kinetic characteristics of the F6a strain on degrading SMX at different concentrations of SMX. With the increase of the initial concentration of SMX, the removal rate of SMX showed a trend of increasing at first and then decreasing. When the initial concentrations of SMX were respectively 1 mg/L, 5 mg/L, 10 mg/L, 50 mg/L, 80 mg/L and 100 mg/L, the removal rates of SMX were respectively 56.27%, 72.14%, 76.95%, 80.17%, 54.45% and 36.12% after 336 hours of degradation. The higher the initial concentration of SMX, the growth and reproduction of the F6a strain will be inhibited, resulting in that the F6a strain has a longer lag phase during the degradation of SMX, thereby causing substrate inhibition effect. The kinetic process of the F6a strain on degrading SMX may be fitted by the pseudo-first-order kinetic equation. The fitting parameters for the kinetic model are shown in Table 4. The SMX degradation rate of the F6a strain was greatly affected by the initial concentration of SMX. The degradation rate constant reached a maximum, which was 1.07×10⁻² h⁻¹, when the initial concentration of SMX was 50 mg/L.

TABLE 4
Fitting parameters of pseudo-first-order kinetic
equation for degradation of SMX by F6a strain

| Concen- | | | | |
|---|---|---|---|---|
| trations | | Degradation | Half- | |
| of SMX | | rate constant | life | |
| (mg · L⁻¹) | Fitting equation | (h⁻¹) | (h) | R² |
| 1 | lnC = −0.0042t + 0.7553 | 0.0042 | 165.00 | 0.9002 |
| 5 | lnC = −0.0079t + 1.5852 | 0.0079 | 87.72 | 0.9875 |
| 10 | lnC = −0.0096t + 2.1271 | 0.0096 | 72.19 | 0.9535 |
| 50 | lnC = −0.0107t + 3.8126 | 0.0107 | 64.77 | 0.9295 |
| 80 | lnC = −0.0050t + 4.3285 | 0.0050 | 138.60 | 0.9723 |
| 100 | lnC = −0.0023t + 4.4409 | 0.0023 | 301.30 | 0.9189 |

### 3.2 Analysis of Mineralization Degree in SMX Degradation Process

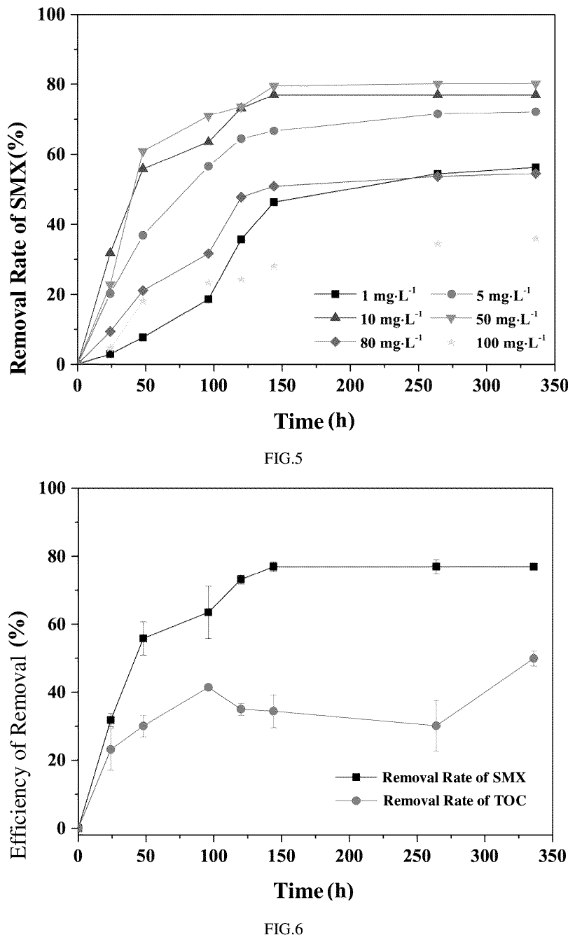

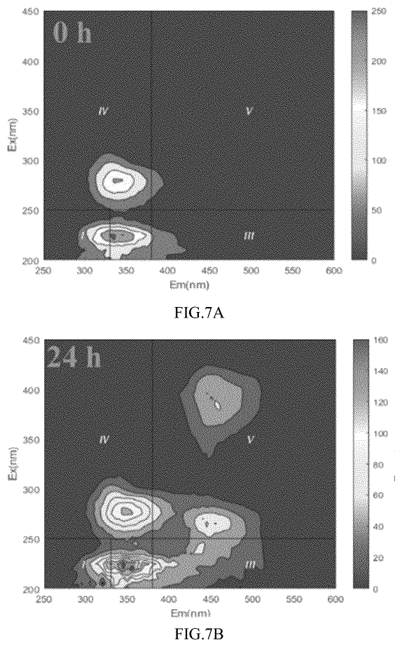

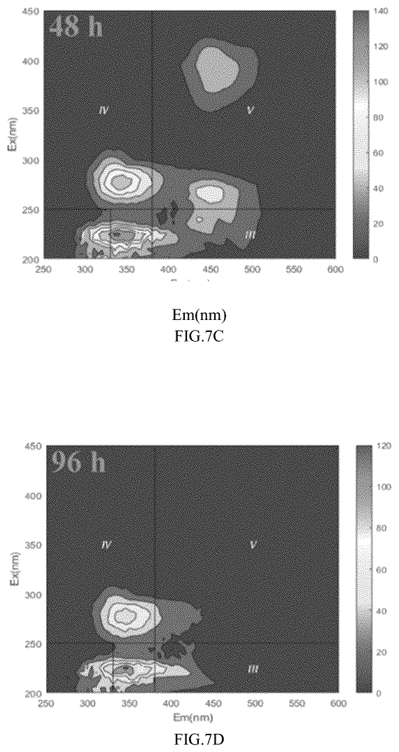

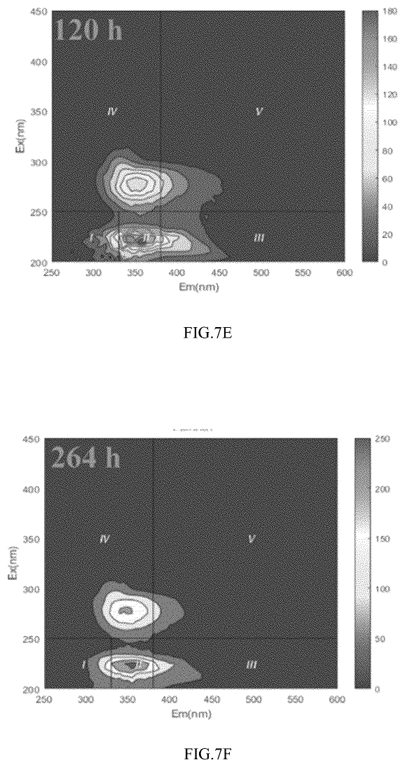

Mineralization degree is an index to evaluate the performance for degrading pollutants. When the initial concentration of SMX was 10 mg/L, the mineralization degree of SMX by the F6a strain was studied. As shown in FIG. 6, with the prolongation of degradation time, the removal rate of SMX gradually decreased, reaching 76.98% at 144 h, but the TOC concentration decreased with a relatively slow trend, with 34.44% of removal rate at 144 h, indicating that the F6a strain could not completely mineralize SMX, and intermediate products of SMX may generated. Further, the removal rate of TOC showed a downward trend at 120 h and reached a minimum at 264 h, with 30.16% of removal rate, which may be due to the reason that the concentration of carbon source available to the F6a strain was low in the late stage of degradation, and intracellular substances in the strain were dissolve out, resulting in a slight increase of the TOC concentration. The reaction process of organic substances in the SMX degradation by the F6a strain was further verified by the three-dimensional fluorescence spectrum analysis. It may be seen from FIG. 7 that the main components in the bacterial solution before the degradation are proteinoids (in zones I and II) and soluble microbial metabolites (in zone IV). With the prolongation of degradation time, the peak intensities of the proteinoids and soluble microbial metabolites were significantly weakened, and humic substances (in region V) were generated during the metabolism of SMX. After 96 hours of degradation, the fluorescence peaks of the humic substances disappeared, which may be caused by the complete metabolism of parts of the intermediates. After 120 hours of degradation, the fluorescence peaks of the soluble microbial metabolites increased slightly, and reached the strongest extent at 264 h, which was consistent with the removal trend of TOC and may be related to the autolysis of bacterial cells. At 336 h, the fluorescence peaks of the proteinoids increased and the fluorescence peaks of the soluble microbial metabolites weakened, which may be due to the reason that SMX was degraded into smaller molecules. Meanwhile, the removal rate of TOC also increased to 49.93%.

### Example 4. Omics Functional Characteristics of F6a Strain

### 4.1 Analysis of Whole Genomics

### 4.1.1 Basic Characteristics of Whole Genomics

The Pseudomonas silesiensis strain A3 (i.e., the F6a strain) was subjected to the whole genome sequencing by using the Next-generation sequencing technology. The total sequence length of the F6a strain is 10264566 bp, which is composed of 106 scaffolds. The F6a strain has an average GC content of 64.05%, 6979 genes, 103 tRNAs, and 5 rRNAs in total over its whole genome.

### 4.1.2 Annotation and Analysis of SMX-Associated Resistance Genes

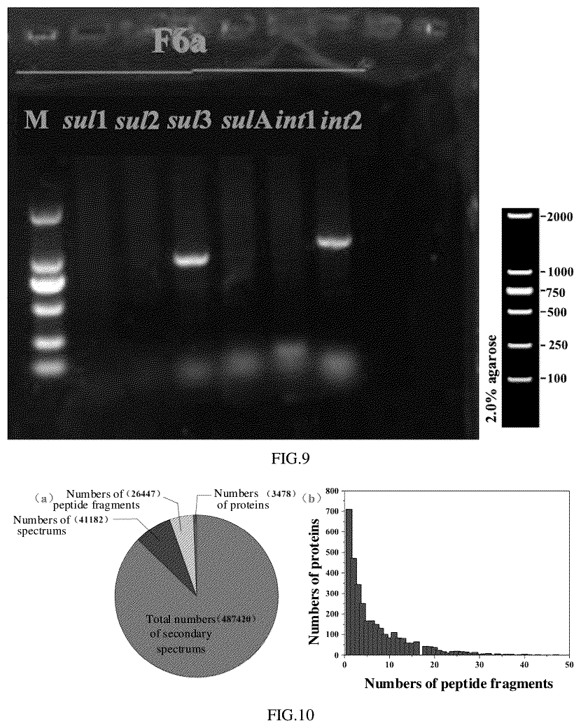

Through the analysis with Comprehensive Antibiotic Research Database (CARD), 577 drug-resistant genes which may be resistant to various antibiotics were identified. Among them, the associated resistance genes (ARGs) having a large number mainly include 77 macrolide antibiotic ARGs, 68 fluoroquinolone antibiotic ARGs, 56 peptide antibiotic ARGs and 52 penicillane ARGs. Only 3 sulfonamide antibiotic ARGs were annotated (FIG. 8a). Of the drug-resistant genome of the F6a strain, antibiotic efflux pump genes dominate absolutely, reaching 63% or more, followed by 6.41% variant or mutated antibiotic resistance genes, 6.41% glycopeptide resistance genes, and 4.91% peptide antibiotic resistance genes. It should be noticed that, no sul1 and sul2 genes were annotated in the F6a strain. For this reason, in the present disclosure, PCR amplification and the electrophoresis detection were carried out on 4 sulfonamide ARGs (i.e., sul1, sul2, sul3 and sulA) and 2 integron genes (i.e., int1 and int2) as target genes. From results of the PCR electrophoresis shown in FIG. 9 (in which several highlighted bands are non-specific fragments), it may be seen that no target fragment bands of the 4 sulfonamide ARGs were detected from the F6a strain, indicating that the F6a strain does not contain the sul1, sul2, sul3 and sulA genes. In addition, a faint band near the target fragment size of the int1 gene indicates that the F6a strain may contain int1 gene. It is worth noting that sul1 and sul2 genes, as representative genes of the sulfonamide ARGs, have a high detection frequency and a high abundance in the environment, and thus there is a high risk. However, no sul1 and sul2 genes were detected from the F6a strain under long-term SMX stress, and SMX was efficiently degraded by the F6a strain. Therefore, the F6a strain has a great application prospect in the treatment of wastewater containing sulfonamide antibiotics.

### 4.2 Proteomic Analysis

Relative quantification of proteins was conducted by the Tandem Mass Tag (TMT) technology. 26447 peptide fragments were identified. Among the identified proteins, 3478 proteins may be quantitatively analyzed. The specific information is shown in FIG. 10. The statistical results showed a total of 296 differential proteins, of which 267 proteins were up-regulated and 29 proteins were down-regulated. The number of up-regulated proteins was more than the number of down-regulated proteins, showing a large difference. Further, the screened differential proteins were compared and analyzed by using the KEGG, GO, COG and PFAM databases to obtain relevant functional annotation information.

4.2.1 Analysis with GO Functional Annotation

The Gene Ontology (GO) database was established by the Gene Ontology Consortium and is applicable to various species, which can be used to define and describe the functions of genes and proteins. The GO database has three primary functions, including biological pathways, cellular components, and molecular functions. According to the results of GO annotation shown in FIG. 11, 10 groups of differential proteins are involved in biological processes, among which 133 proteins are involved in the cellular process and 177 proteins are involved in the metabolic process respectively, accounting for the majority. Analysis of the cellular components showed that differential proteins mainly found in the cellular anatomical entities with 138 proteins and the protein-containing complexes with 23 proteins. There are 10 groups of differential proteins clustered in molecular functions, and most of the differential proteins are involved in the catalytic activity and the binding, among which 142 proteins, including 127 up-regulated proteins, are involved in the catalytic activity, and 133 proteins, including 120 up-regulated proteins, are involved in the binding, indicating that SMX may improve the binding and catalytic activity of cells in the F6a strain, thereby facilitating the degradation of SMX.

### 4.2.2 Analysis of KEGG Pathway

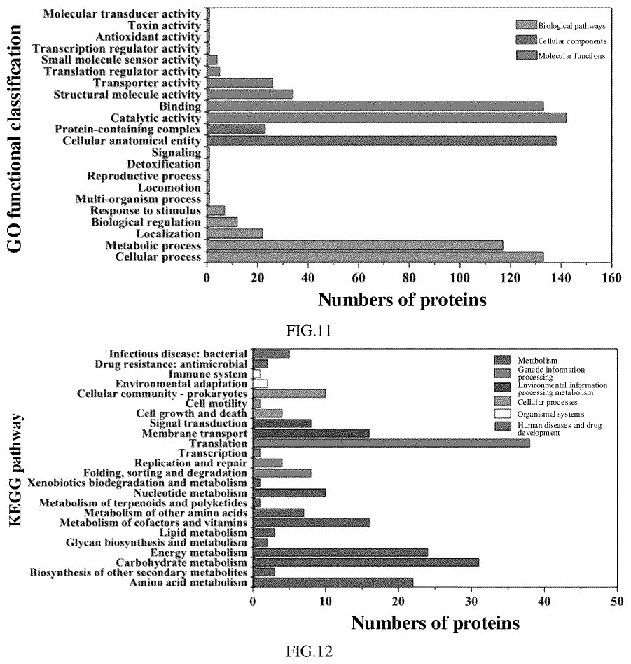

The metabolism pathways of SMX-induced differential proteins and the relationship between individual metabolism pathways were analyzed and studied through KEGG pathway. The results are shown in FIG. 12. The differential proteins are involved in 24 signaling pathways including two metabolic pathways, four genetic information processing, two environmental information processing metabolic pathways, three cellular process pathways, two organismal system pathways, and two human disease and drug development pathways. The metabolic pathways mainly include pathways for carbohydrate metabolism, energy metabolism, amino acid metabolism, metabolism of cofactors and vitamins, and nucleotide metabolism, with 28, 23, 19, 15 and 10 up-regulated proteins respectively. Functional genes involved in the carbohydrate metabolism accounted for the highest proportion, which may be related to the characteristics that the F6a strain utilizes SMX as a carbon source for growth and reproduction.

### 4.2.3 Analysis of COG Annotation

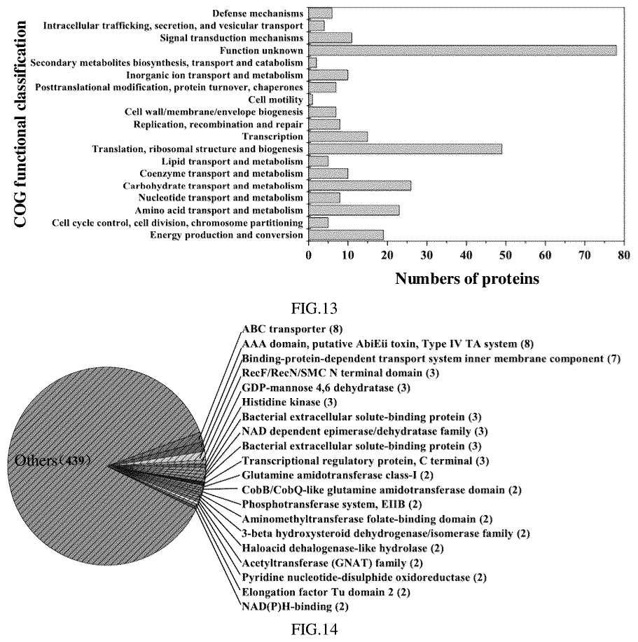

The COG database is constructed based on the classification of the coding proteins of the complete genomes of bacteria, algae and eukaryotes, which are divided into 26 categories depending on their functions, according to their phylogenetic relationship. A total of 294 differential proteins from the F6a strain were annotated into 19 functions in the COG database (FIG. 13). Except for the classification of 78 proteins with unknown functions, the differential proteins corresponding to the functions annotated by a large number of proteins include: 49 differential proteins related to the translation, ribosomal structure and biogenesis; 26 differential proteins related to the carbohydrate metabolism; 23 differential proteins related to the amino acid transport and metabolism; and 19 differential proteins related to the energy production and conversion. There are 48 up-regulated proteins related to the translation, ribosomal structure and biogenesis, indicating that the efficiency of translation activity of the F6a strain under the stress of SMX is improved, which promotes the synthesis of proteins related to the degradation of organic substances, thereby facilitating the degradation of SMX. In addition, synthetic proteins were overexpressed in cells, which may possibly to compensate for damaged proteins.

4.2.4 Analysis with PFAM Annotation

The PFAM database is a collection of protein families. The families and structural domains of the proteins related to the COG functions and KEGG pathways may be completely and accurately classified according to the protein sequence alignment, and the functions of proteins may be further analyzed according to the structural domains of proteins. The differential proteins were annotated based on the PFAM database to obtain relevant biological functions. The results are shown in FIG. 14. In the PFAM database, the matched differential proteins were divided into 427 categories according to their functions, among which the greatest number of differential proteins involved in the amino acid transport and synthesis (such as, ABC transporter) and ATP hydrolysis (with 8 differential proteins,) followed by 7 differential proteins that involved in the transport process in the inner membrane.

The proteins of the F6a strain with a large difference in expression level after SMX treatment are listed in Table 5. For the treatment group, the differential protein DUF5302 family protein exhibited the highest expression level, which was about 10.89 times higher than that of the control group. DSBA oxidoreductase had the second highest expression level, which was about 10.22 times higher than that of the control group. The main function of the DSBA oxidoreductase is to participate in the biosynthesis, transportation and catabolism of secondary metabolites. The DSBA protein can directly catalyze the substrate to form disulfide bonds, with strong catalytic activity. In the catalytic reaction, the DSBA protein allows formation of an intermolecular covalent disulfide bond between the cysteine at the position 30 of the active center and —SH of the C10 molecule of the intermediate products of the SMX metabolism. Besides, tryptophan synthase subunit alpha is involved in the transport and metabolism of amino acids, and the tryptophan synthase subunit alpha can encode independently, and have the ability of catalyzing the reaction alone or regulating the catalytic reaction. Sodium-translocating pyrophosphatase and respiratory nitrate reductase subunit gamma, as other up-regulated proteins, have expression levels which are 7.45 times and 7.14 times higher than and greatly different from those in the control group, respectively. The sodium translocating pyrophosphatase mostly found on the cell membrane, and can participate in the hydrolysis of SMX, the transmembrane transport of active ions, and the production and transport of energy in terms of molecular functions. The respiratory nitrate reductase subunit, as an oxidoreductase, can function as a donor and act on nitrogen-containing compounds such as SMX and its intermediate products, and can also participate in catalytic reactions and production and conversion of energy.

In terms of particular down-regulated proteins, for the treatment group, the differential protein MtnX-like HAD-IB family phosphatase exhibited the lowest expression level, which was 0.24 times lower than that of the control group. The MtnX-like HAD-IB family phosphatase can remove phosphate groups on substrate molecules through the catalytic hydrolysis of phosphomonoester, generate phosphate ions and free hydroxyl groups, and participate in the transport and metabolism of amino acids. The expression levels of sugar ABC transporter permease and branched-chain amino acid transport system II carrier protein were both 0.27 times lower than those of the treatment group. They mainly involved in transmembrane transport of ions or molecules, and also involved in the transport and metabolism of carbohydrates, and the transport and metabolism of amino acids. Therefore, SMX may inhibit the transport and permeation of parts of nutrients.

TABLE 5
Parts of proteins with differential expression between
treatment group and control group of F6a strain

| ID | Protein name | FC |
|---|---|---|
| gene6494 | DUF5302 family protein | 10.89 |
| gene0618 | DSBA oxidoreductase | 10.22 |
| gene0847 | tryptophan synthase subunit alpha | 9.80 |
| gene4124 | sodium-translocating pyrophosphatase | 7.45 |
| gene0552 | respiratory nitrate reductase subunit gamma | 7.14 |
| gene4317 | heavy metal response regulator transcription factor | 0.36 |
| gene3466 | hypothetical protein OU5_2913 | 0.32 |
| gene4506 | branched-chain amino acid transport system II carrier | 0.27 |
| | protein | |
| gene5187 | sugar ABC transporter permease | 0.27 |
| gene5522 | MtnX-like HAD-IB family phosphatase | 0.24 |

Note:
FC(CT/CK) indicates the differential expression fold of proteins between the treatment group (CT) and the control group (i.e., control check, CK).

### Example 5. Analysis of Metabolic Pathways of SMX

In the present disclosure, the intermediate products of SMX were analyzed by HPLC/MS/MS at 24 h, 48 h, 96 h and 144 h when the F6a strain degraded SMX, so as to further analyze the metabolic pathways of SMX. The genes and proteins that may be involved in the pathways of SMX degradation were screened through results of the whole genome and proteome annotation, focusing on differentially expressed proteins, so as to find out the useful genes (or gene clusters) involved in the efficient degradation of SMX by the F6a strain through the differential expression of different functional proteins, and further to explain and improve the degradation pathways and degradation mechanisms of SMX. As shown in Table 6, the expression of proteins encoded by various genes, such as ribosomal proteins, the tricarboxylic acid cycle-related enzymes, etc., was significantly up-regulated, indicating that the energy metabolism of the F6a strain was significantly enhanced under the stress of SMX, and stress responses appeared to improve bacterial survivability and the degradation of SMX. Transporters and ATPases (such as ABC transporter permease, cell division ATP-binding protein FtsE and ABC transporter ATP-binding protein, etc.) were also significantly up-regulated, which maintain their own life activities by regulating transport through cell membrane and hydrolyzing ATP to release energy.

TABLE 6
Significantly up-regulated top 20 differential proteins in proteomics analysis of F6a strain

| Protein ID | Protein name | FC |
|---|---|---|
| gene0181 | malate dehydrogenase [Cellulomonas soli] | 2.07 |
| gene0377 | dTDP-glucose 4,6-dehydratase [Cellulomonas soli] | 2.29 |
| gene0552 | respiratory nitrate reductase subunit gamma [Cellulomonas soli] | 7.14 |
| gene0618 | DSBA oxidoreductase [Cellulomonas soli] | 10.22 |
| gene0655 | LLM class F420-dependent oxidoreductase [Cellulomonas soli] | 2.11 |
| gene0847 | tryptophan synthase subunit alpha [Cellulomonas soli] | 9.80 |
| gene0900 | type 1 glyceraldehyde-3-phosphate dehydrogenase [Cellulomonas | 2.56 |
| | oligotrophica) | |
| gene0924 | alpha/beta hydrolase [Cellulomonas timonensis] | 2.56 |
| gone1110 | FAD-dependent oxidoreductase [Cellulomonas soli] | 2.49 |
| gene1224 | quinone-dependent dihydroorotate dehydrogenase [Cellulomonas soli] | 2.16 |
| gene1751 | NADH-quinone oxidoreductase subunit J [Cellulomonas soli] | 2.95 |
| gene1753 | NADH-quinone oxidoreductase subunit NuoH [Cellulomonas soli] | 2.56 |
| gene2058 | alpha/beta fold hydrolase [Cellulomonas soli] | 2.50 |
| gene2832 | family 43 glycosylhydrolase [Cellulomonas soli] | 2.11 |
| gene2862 | N-dimethylarginine dimethylaminohydrolase [Cellulomonas soli] | 2.41 |
| gene3970 | dihydroxy-acid dehydratase [Cellulomonas soli] | 2.08 |
| gene4012 | 3-isopropylmalate dehydratase small subunit [Cellulomonas soli] | 2.35 |
| gene4124 | sodium-translocating pyrophosphatase [Cellulomonas soli] | 7.45 |
| genc6494 | DUFS302 family protein [Cellulomonas soli] | 10.89 |
| gene6530 | NAD-dependent succinate-semialdehyde dehydrogenase [Cellulomonas soli] | 4.62 |

Only sadA, sadB and sadC gene clusters were found to degrade sulfonamides antibiotics in published literatures. The sadA and sadC will first attack the sulfonamide molecule to produce 4-aminophenol, and the 4-aminophenol is further converted to 1,2,4-triphenol through the degradation by the sadB and sadC. In the present disclosure, the raw data of whole genome was aligned with the reported sequences of sadA, sadB and sadC genes and amplification was carried out three times with the designed PCR primers (FIG. 15). No genes having high similarity to the sadA, sadB and sadC gene clusters were detected in both ways. Therefore, it is inferred in the present disclosure that there may exist a new SMX degradation mechanism in the SMX degradation process by the F6a strain.

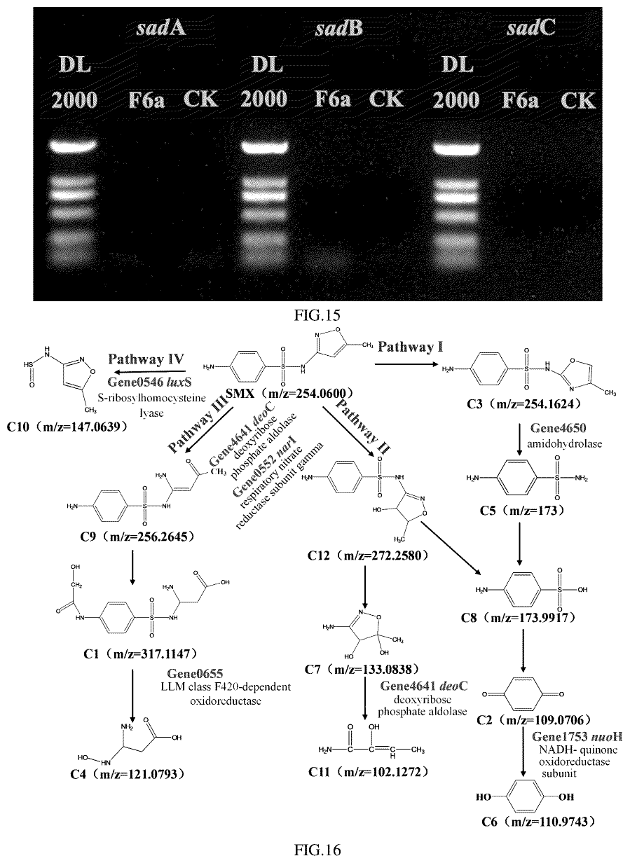

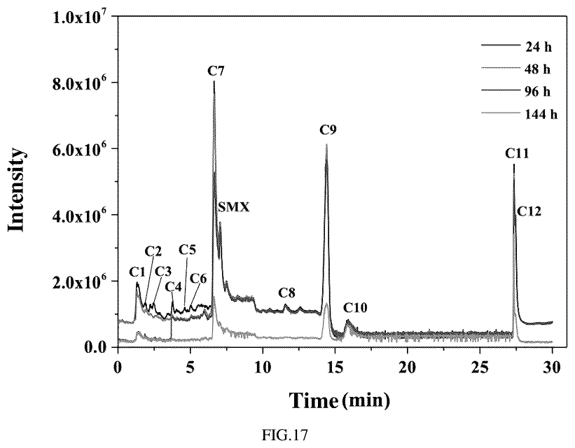

During the SMX degradation by the F6a strain, four metabolic pathways (FIG. 16) and 12 intermediate products were found (FIG. 17). The peaks of the intermediate products appeared at 1.318 min, 2.476 min, 3.762 min, 6.653 min, 11.575 min, 14.412 min, 15.877 min, 27.361 min or 27.451 min respectively. The ion peaks of the intermediate products were respectively as follows: m/z=317.1147 (C1), m/z=109.0706(C2), m/z=254.1624(C3), m/z=121.0793(C4), m/z=173.1167 (C5), m/z=110.9743(C6), m/z=133.0838(C7), m/z=173.9917(C8), m/z=256.2645 (C9), m/z=147.0639(C10), m/z=102.1272(C11), and m/z=272.2580(C12). The metabolism was mainly carried out according to the pathway III. In pathway I, C3 is generated due to the isomerization of the isoxazole ring. The amidohydrolase encoded by Gene 4650 has strong hydrolase activity and can catalyze the hydrolysis of various bonds, such as C—O bond, C—N bond, C—C bond, phosphate anhydride bond, etc., mainly acting on the C—N bond other than peptide bond, which may lead to the generation of C5 (i.e., sulfanilamide). However, due to the limitation of the next-generation sequencing technology, the gene encoding this protein in the whole genome has not been detected. Subsequently, the amino in the sulfonyl group is substituted with a hydroxyl group to generate C8. In addition, the NADH-quinone oxidoreductase subunit encoded by the Gene 1753 nuoH gene can catalyze the conversion of C2 (i.e., benzoquinone) into C6 (i.e., p-aminophenol), and C6 cannot be further mineralized into gas.

In Pathway II, the C═C double bond of the isoxazole ring undergoes an addition reaction first and then is substituted with a hydroxyl group to generate C12, after which the sulfanilamido group is subjected to the acidification and hydrolysis, leading to the breakage of the S—N bond to generate two forms of intermediates respectively centered on isoxazole ring and aniline, such as C8 (i.e., 4-aminobenzenesulfonic acid) and C7 (FIG. 16). Although 3-amino-5-methylisoxazole was not found in the present disclosure, the structures of most metabolites showed changes in the isoxazole ring. C7 may be the result of electrophilic substitution of the C═C double bond of 3-amino-5-methylisoxazole. C7 is then further degraded by microorganisms and is ring-opened to generate C11. This process may involve the catalysis of deoxyribose phosphate aldolase (2-deoxyribose-5-phosphate aldolase) encoded by the Gene4641 deoC gene, which results in the cleavage of the isoxazole ring. Studies have shown that the deoxyribose phosphate aldolase can catalyze the reversible aldol reaction between acetaldehyde and D-glyceraldehyde 3-phosphate to generate 2-deoxy-D-ribose 5-phosphate.

In Pathway III, the isoxazole ring of SMX is oxidized by the respiratory nitrate reductase encoded by the Gene0552 narI gene, leading to the cleavage of the N—O bond to generate C9. The relative intensity of this peak decreases significantly at 336 h. C1 may be derived from the acetylation and the hydroxylation of the amino group on the benzene ring. Both ammonia-oxidizing bacteria and ammonia-oxidizing archaea can oxidize ammonia to hydroxylamine through ammonia monooxygenase (AMO). C4 may be generated by catalyzing the S—N bond of sulfonamide group of C1 by the monooxygenase LLM class F420-dependent oxidoreductase encoded by an unknown gene Gene0655 in the co-metabolism process. The redox activity of the AMO can act on paired donors and bind or reduce molecular oxygen. Studies have shown that when the effects of different ammonia oxidants on the biological metabolism of sulfonamide antibiotics are investigated, it is found that the amine group of sulfonamide antibiotics may be hydroxylated via the hydroxylamine reaction under the action of AMO.

In pathway IV, it was also detected that the intermediate product C10 was generated by the breakage of the S—C bond, which was dominated by the S-ribosylhomocysteine lyase encoded by the Gene0546 luxS gene. Gene0546 luxS can regulate the quorum-sensing mechanism, which mainly involves in the synthesis of auto-inducer 2 (AI-2) secreted by bacteria, and is used to convey the cell density and the metabolic potential of environment, thereby regulating the gene expression in response to changes in the cell density. Studies have shown that the quorum-sensing mechanism widely exists in pathogenic bacteria and is closely related to the infection process, the expression of pathogenic genes and the final pathogenic process. However, the absorption peak of C10 did not change during the whole metabolic process, indicating that C10 was not further mineralized with the prolongation of degradation time. It is worth noting that no absorption peaks of C3 and C4 were detected at 264 h, and no absorption peak of C8 was detected at 336 h, indicating that C3, C4 and C8 were completely mineralized into inorganic small molecular substances such as CO₂, H₂O, SO₄₂ and the other.

In addition, it is worth noting that the MarR family transcriptional regulator, as an expression protein of pathogenic bacteria that regulates physiological pathways such as virulence factors, is significantly up-regulated. In addition, a series of proteins in the anti-virus system such as PRD domain-containing protein encoded by Gene4896 and antitoxin encoded by Gene3029 were also significantly up-regulated (Table 7), which can adjust the ratio of toxin to antitoxin components of II-TA system, and can allow the formation of bacteria or the restoration of semi-dormant stock bacteria. However, antibiotics have no effect on these proteins and thus the bacterial cells develop drug resistance. Not only that, the TA system also has many functions, such as inducing cell dormancy to adapt to various stress responses, inhibiting phage, regulating gene expression or the like. In addition, it was found that the penicillin-binding protein encoded by Gene3153 gene was up-regulated, which can selectively and non-covalently interact with penicillin, an antibiotic containing condensed β-lactam-thiazolidine ring. The penicillin-binding protein may also be a potential protein involved in SMX degradation. Although the enzyme-catalyzed reactions involved in some degradation pathways may be annotated through the whole genome and the proteome, the entire degradation process, including the hydroxylation process in pathway I and pathway II and the isomerization of the isoxazole ring in pathway III, cannot be fully presented due to the complexity of microbial metabolism.

TABLE 7
Genes inferred to involve in SMX degradation based on information of genome and proteome

| | | | | Significantly upregulated | |
|---|---|---|---|---|---|
| Gene ID | Gene name | FC | Database | functional proteins and hosts | FDRc |
| Gene4641 | deoC | 2.00 | UniprotKB/ | Deoxyribose-phosphate aldolase | High |
| | | | Swiss-Prot | [Cellulomonas soli] | |
| Gene0552 | narI | 7.14 | Swiss-Prot | Respiratory nitrate reductase subunit | High |
| | | | | gamma | |
| | | | | [Cellulomonas soli] | |
| Gene0655 | — | 2.10 | Nonredundant (NR) | LLM class F420-dependent | High |
| | | | | oxidoreductase | |
| | | | | [Cellulomonas soli] | |
| Gene0546 | luxS | 2.13 | NR | S-ribosylhomocysteine lyase | High |
| | | | | [Cellulomonas soli] | |
| Gene4650 | — | 2.01 | NR | Amidohydrolase | High |
| | | | | [Cellulomonas soli] | |
| Gene1753 | nuoH | 2.56 | NR | NADH-quinone oxidoreductase subunit | High |
| | | | | nuoH | |
| | | | | [Cellulomonas soli] | |
| Gene4896 | licT | 2.97 | Swiss-Prot | PRD domain-containing protein | High |
| | | | | [Demequina salsinemoris] | |
| Gene3029 | — | 2.09 | NR | Antitoxin | High |
| | | | | [Cellulomonas sp. Root137] | |
| Gene3153 | — | 2.40 | NR | penicillin-binding protein | High |
| | | | | [Cellulomonas soli] | |
| Gene6410 | — | 2.94 | NR | MarR family transcriptional regulator | High |
| | | | | [Actinotalea sp. HO-Ch2] | |

Note:
“—” represents unannotated. FDRc (FDR Confidence) represents the evaluation of the confidence level of proteins, which is divided into High, Medium and Low.

### 6. Summary

In the present disclosure, useful SMX-degrading strains in the bioelectrochemically enhanced constructed wetland (BE-CW) were screened out by the plate streaking method. The F6a strain having a high efficiency on degrading SMX was subjected to morphological characterization. The SMX-degrading performance of the F6a strain was verified through degradation kinetic experiments. Intermediate products that may be generated during the degradation process were analyzed by the UPLC-QTOF-MS. The regulation effects of useful genes in the degradation process were analyzed through genomics and proteomics. The main conclusions are as follows.
1) A total of 47 strains were identified after enrichment in the R2A medium containing 1 mg/L, 10 mg/L and 100 mg/L of SMX and isolation. These strains belong to 28 genera including Bacillus, Pseudomonas, Methylotenera or the like, and belong to 4 phyla including Proteobacteria, Firmicutes, Bacteroidetes, Actinobacteria or the like. Most strains belong to the Proteobacteria phylum. Bacillus cereus strain IAM 12605, Bacillus cereus ATCC 1457, Methylotenera mobilis JLW8, Microbacterium flavescens strain IFO 15039 and Pseudomonas silesiensis strain A3 are useful SMX-degrading bacteria in the bioelectrochemically enhanced constructed wetlands. For both the MFC-CW and the EC-CW, the types of useful SMX-degrading strains are basically the same, in which the F6a strain, as an electrochemically active bacterium, exhibited the best degradation effect. According to the 16S rDNA identification, the F6a strain is identified to belong to Proteobacteria phylum, Gammaproteobacteria class, Pseudomonadales order, Pseudomonadaceae family, and Pseudomonas genus.
2) The kinetic process of SMX degradation by the F6a strain conforms to the pseudo-first-order kinetic equation. The degradation rate is greatly affected by the initial concentration of SMX. The degradation rate constant is 0.0023 h 1 to 0.0107 h 1. When the initial concentration of SMX is 10 mg/L, the removal rate of TOC could reach 49.93%. With the prolongation of degradation time, the peak intensity of proteinoids and soluble microbial intermediates weakens obviously.
3) The whole genome of the F6a strain has 106 scaffolds, a sequence length of 10264566 bp, and an average GC content of 64.05%, 6979 genes, 103 tRNAs, and 5 rRNAs in total over the whole genome. Through the CARD analysis, a total of 577 drug-resistant genes which may be resistant to various antibiotics were identified. The antibiotic efflux pump genes are the main mechanism of resistance to SMX by the F6a strain. Through the proteomic analysis, a total of 296 differential proteins are found, in which 267 differential proteins are up-regulated, and 29 differential proteins are down-regulated. These differential proteins are mainly involved in cellular processes and metabolic processes.
4) During the SMX degradation by the F6a strain, 4 metabolic pathways and 12 intermediate products are found. Gene4641 deoC, Gene0552 narI, Gene0546 luxS, Gene1753 nuoH, Gene0655 and Gene4650 are six useful genes involved in the SMX degradation. The SMX degradation process is performed mainly based on the pathway III. Firstly, the respiratory nitrate reductase encoded by Gene0552 narI oxidizes the isoxazole ring of SMX, leading to the cleavage of the N—O bond to generate C9. 4-N-(hydroxymethyl carboxyl)-N-(3-amino-5-carboxy) benzenesulfonamide (i.e., C1) is generated by the acetylation and hydroxylation. The S—N bond of sulfanilamido group of C1 is catalyzed by the monooxygenase LLM class F420-dependent oxidoreductase to generate 3-amino-5-carboxyl hydroxylamine (i.e., C4).

The technical features of the above-mentioned embodiments may form any combination. For concise description, all possible combinations of the technical features in the above-mentioned embodiments are not described. However, as long as the combinations of these technical features are not contradicted, they should be considered as within the scope of this specification.

The above-mentioned embodiments merely express several implementation modes of the present disclosure and are described in relatively specific and detailed descriptions, but they should not be construed as limiting the scope of the present disclosure. Several modifications and improvements may be made by those skilled in the art without departing from the concept of the present disclosure, and these all belong to the protection scope of the present disclosure. Therefore, the protection scope of the present disclosure should be based on the appended claims.

1. An isolated Pseudomonas silesiensis strain, which is deposited in the China Center for Type Culture Collection on Apr. 6, 2021 with a deposit number of CCTCC No: M2021338.

2. A composition comprising the strain according to claim 1.

3. The composition according to claim 2, wherein the composition is in a form of liquid, or frozen or dry powder.

4. The composition according to claim 3, wherein the composition comprises an adjuvant.

5. The composition according to claim 4, wherein the adjuvant comprises one or more of sodium dodecylbenzenesulfonate, sodium butylnaphthalenesulfonate, trehalose, glycerin, sodium lignosulfonate, polycondensate of sodium alkylnaphthalenesulfonate, nicotinic acid, alcohol, buffer salt, sodium chloride, amino acid, vitamins, protein, polypeptide, polysaccharide or monosaccharide, yeast extract, white carbon black, tea saponin, or skimmed milk.

6. The composition according to claim 3, wherein when the composition is in the form of frozen or dried powder, the composition further comprises a solid carrier; and
the solid carrier comprises one or more of peat, turf, talc, lignite, pyrophyllite, montmorillonite, alginate, filter press slurry, sawdust, perlite, mica, silica, quartz powder, calcium-based bentonite, vermiculite, kaolin, light calcium carbonate, diatomaceous earth, medical stone, calcite, zeolite, white carbon black, fine sand and clay.

7. A method for culturing the strain according to claim 1, comprising culturing the Pseudomonas silesiensis strain in a R2A medium.

8. A method for degrading sulfamethoxazole in water, soil or sludge comprising adding the strain according to claim 1 to the water, soil or sludge.

9. The method according to claim 8, wherein the in water, soil or sludge was contaminated by antibiotics.

10. The method-use according to claim 8, wherein the strain cooperates with a microbial fuel cell to degrade sulfamethoxazole.

11. A method for degrading sulfamethoxazole in water, soil or sludge comprising adding the composition according to claim 2 to the water, soil or sludge.

12. The method according to claim 11, wherein the water, soil or sludge was contaminated by antibiotics.

13. The method according to claim 11, wherein the composition cooperates with a microbial fuel cell to degrade sulfamethoxazole.


SEQUENCE LISTING (1 sequence)

SEQ ID NO: 1 - DNA, 899 bp, Pseudomonas silesiensis
gcgggggggcgctaccatgcagtcgagcggcagcacgggtacttgtacctggtggcgagcggcggacgggtgagtaatgcctaggaatctgcctggtagtgggggataacgctcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtatctgttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgacaagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcgg